(19) United States
(12) Patent Application Publication
(10) Pub. No.: US 2010/0036400 A1
(43) Pub. Date: Feb. 11, 2010

(54) Vascular anastomosis device

(76) Inventor: Emad T. Aboud, Swaida (SY)

Correspondence Address: WRIGHT, LINDSEY & JENNINGS LLP, 200 WEST CAPITOL AVENUE, SUITE 2300, LITTLE ROCK, AR, 72201-3699, US

(21) Appl. No.: 12/583,423

(22) Filed: Aug. 20, 2009

Related U.S. Application Data
(63) Continuation-in-part of application No. 12/228,222, filed on Aug. 11, 2008.

(51) Int. Cl. A61B 17/11

(52) U.S. Cl. 606/153

(57) ABSTRACT

A device for creating an end-to-side anastomosis between a graft vessel and a recipient vessel having an extravascular member, such as an upper ring, an intravascular member, such as a lower ring, and means to fix the intravascular member in the recipient vessel, such as a handle with a resilient element biasing the extravascular member and the intravascular member together. A graft vessel is attached to the intravascular member around an edge of an opening in the intravascular member. Other means than a biasing resilient element may be employed to fix the intravascular member in the recipient vessel which do not require an extravascular member, such as expandable cylinders. In other embodiments, means may be employed to insert the intravascular member into the recipient vessel such as delivery systems that open an arteriotomy while simultaneously delivering the intravascular member into the recipient vessel.

### CROSS-REFERENCE TO RELATED APPLICATIONS

This application is a continuation-in-part of co-pending U.S. patent application Ser. No. 12/228,222 filed Aug. 11, 2008, the disclosure of which is incorporated herein by reference.

### STATEMENT REGARDING FEDERALLY SPONSORED RESEARCH OR DEVELOPMENT

Not applicable.

### BACKGROUND OF THE INVENTION

1. Field of the Invention

The present invention relates to devices for vascular anastomosis, and in particular, to devices for end-to-side vascular anastomoses.

2. Brief Description of the Related Art

Devices for assisting in vascular anastomosis are known but most of them are complicated and have more than one part which makes the anastomosis more complicated to perform.

The limitations of the prior art are overcome by the present invention as described below.

### BRIEF SUMMARY OF THE INVENTION

The present invention is a clip made of metal or similar materials for use in end-to-side anastomoses, i.e., in connecting the end of a homogeneous graft vessel or artificial graft to the side of a recipient vessel in different parts of a human or animal body. The clip comprises a handle and two rings, a lower ring and an upper ring. The two rings are closely juxtaposed when the clip is closed. The handle may be similar to but smaller than the aneurysm clips known in the art, for example, the aneurysm clip disclosed in U.S. Pat. No. 4,765,335, the disclosure of which is incorporated herein in its entirety.

The handle may fit into a clip applier that facilitates the application of the clip as described below. The handle and the clip applier may be of different shapes depending on the various applications of the clip.

The rings may also have different shapes and sizes to accommodate different kinds of anastomoses and different vessels of all sizes in all parts and cavities of the human or animal body, including without limitation the brain, thorax, abdomen, pelvis, neck and limbs. The rings may be of various shapes, including but not limited to circular or elliptical. When the clip is used with small vessels, for example in cerebrovascular anastomoses within the skull, it is desirable that the size of the lower ring and the handle be as thin as possible.

To ensure a firm grasp of the rings on the vessel walls, the inner surface of each of the rings, i.e., the surface facing the other ring, may be provided with a rough surface or the surface of one ring may be provided with spines that fit into holes in the facing surface of the other ring. The handle may additionally urge the two rings together by, for example, a resilient element such as a spring.

The clip of the present invention has a wide range of uses all over the body. The present invention also has the benefits of shortening surgery time and minimizing occlusion time during regular anastomoses as compared to using other complicated devices known in the prior,art. The different sizes and shapes of the clip widen the range of its use. In addition the embodiment of the clip incorporating a graft vessel made of synthetic material will provide the vascular surgeon with a wide range of choices making it a benefit to vascular surgery. As compared to the prior art, the present invention is simpler, easier to use, composed of one piece, and it can be used on vessels of 2 mm up to the largest vessels in the body.

As described above, certain embodiments of the present invention comprise an extravascular member such as an upper ring, an intravascular member such as a lower ring, and means to fix the intravascular member in the recipient vessel such as a handle with a resilient element biasing the extravascular member and the intravascular member together. A graft vessel is attached to the intravascular member around an edge of an opening in the intravascular member. When means other than a biasing resilient element are employed to fix the intravascular member in the recipient vessel, certain embodiments of the present invention may not require an extravascular member. In other embodiments of the present invention, means may be employed to insert the intravascular member into the recipient vessel, for example, delivery systems that open an arteriotomy while simultaneously delivering the intravascular member into the recipient vessel.

These and other features, objects and advantages of the present invention will become better understood from a consideration of the following detailed description of the preferred embodiments and appended claims in conjunction with the drawings as described following:

### BRIEF DESCRIPTION OF THE SEVERAL VIEWS OF THE DRAWING

FIG. 1 is a perspective view of one embodiment of the present invention in which the clip is formed from a single length of wire and a resilient element in the form of a coil spring is formed into the handle of the clip.

FIG. 2 is a right side elevation view of the embodiment of FIG. 1.

FIG. 3 is a top plan view of the embodiment of FIG. 1.

FIG. 4 is a cross sectional detail of the relationships among the rings and the offset segments taken along the line 4-4 of FIG. 3.

FIG. 5 is a top plan view of an embodiment of the present invention illustrating in phantom outline various sizes of substantially circular rings.

FIG. 6 is a top plan view of an embodiment of the present invention illustrating in phantom outline various sizes of wide elliptical rings.

FIG. 7 is a top plan view of an embodiment of the present invention illustrating in phantom outline various sizes of narrow elliptical rings.

FIG. 8 is an elevation view of an alternative embodiment of the present invention in which a resilient element in the form of a segment of elastic material is disposed between the arms of the handle.

FIG. 9 is an elevation view of an alternative embodiment in which the clip is formed from a single length of wire with the two arms of the handle connected by a simple half circle. A resilient element in the form of a coil spring is disposed between the arms of the handle.

FIG. 10 is an elevation view of an alternative embodiment in which the two arms of the handle are pivotally connected.

FIG. 11 is an elevation view of an alternative embodiment having pivotally connected arms as shown in FIG. 10 and a coil spring between the arms as shown in FIG. 9.

FIGS. 12-15 are views of an embodiment of the present invention being employed to attach a graft vessel to a recipient vessel. FIGS. 12-14 are perspective views showing the graft vessel sutured to the lower ring (FIG. 12), the rings opened to allow the lower ring to be inserted into an arteriotomy in the recipient vessel (FIG. 13) and the rings closed to complete the anastomosis (FIG. 14). FIG. 15 is a cross sectional detail along the line 15-15 of FIG. 14.

FIG. 16 is an elevation view of two clips being employed to attach a graft vessel between a donor vessel and a recipient vessel.

FIG. 17 is an elevation view of a clip being employed to attach an artificial aneurysm to a recipient vessel for surgical training purposes.

FIGS. 18-19 are elevation views of two embodiments of a clip applier.

FIG. 20 is a perspective view of the upper and lower rings with their respective inner surfaces roughened.

FIGS. 21A-E are elevation views of various embodiments of the handle.

FIG. 22 is an elevation view similar to that of FIG. 16 showing a graft with corrugations that allow it to be stretched to a desirable length between a donor vessel and a recipient vessel.

FIG. 23 is a perspective view of an embodiment of the present invention showing a handle with plates having an indentation for ease of grasping by a clip applier, rings with upwardly and outwardly extending lips, and rings that conform to the shape of the recipient vessel.

FIG. 24 is a perspective view of an alternative embodiment of FIG. 23 in which the intravascular member is a hemi-cylinder.

FIG. 25 is perspective view of an alternative embodiment of FIG. 24 in which the extravascular member is a hemi-cylinder.

FIG. 26 is a perspective view of an alternative embodiment of FIG. 23 in which the intravascular member is a compressible scroll-like body.

FIG. 27A is a perspective view of an alternative embodiment of FIG. 26 in which the extravascular member is a hemi-cylinder.

FIG. 27B is a cross-sectional view along the line 27B-27B of FIG. 27A showing the attachment of wires between the scroll-like body and the extravascular member, in this case a ring.

FIG. 28A is a perspective view of an alternative embodiment of FIG. 26A in which the extravascular member is eliminated and the handle includes two arms that are attached to the exterior of the scroll-like body, one arm to either side so that compression of the handle compresses the body.

FIG. 28B is a perspective view of the alternative embodiment of FIG. 28A showing the device inserted through an arteriotomy into the interior of the recipient vessel and allowed to expand against the walls of the recipient vessel to fix the body into position.

FIG. 29A is a perspective view of a further embodiment of the present invention in which the graft vessel is connected to both a recipient vessel and a donor vessel by means of T-shaped ends holding in horizontal short shafts an intravascular member which is a stent-like expandable cylinder.

FIG. 29B is a perspective view of the embodiment of FIG. 29A showing the device attached to a recipient vessel and a donor vessel.

FIG. 30A is a perspective view of a delivery needle for inserting an intravascular member into a recipient vessel.

FIG. 30B is a cross-sectional view of the delivery needle of FIG. 30A along the line 30B-30B of FIG. 30A showing the boat shape of the needle and the two sharp upper edges of the anterior portion of the needle that serve to open an arteriotomy in the wall of the recipient vessel while simultaneously delivering the intravascular member inside the vessel.

FIG. 30C is a perspective view of the delivery needle of FIG. 30A shown opening an arteriotomy in a recipient vessel while delivering an intravascular member of the embodiment of FIGS. 29A and 29B.

FIG. 31 is a perspective view of an alternative embodiment of a delivery needle having a boat shape with sharp upper cutting edges to make an arteriotomy when inserted into the recipient vessel. The needle has a hollow interior portion that may receive the intravascular member so that the arteriotomy and the delivery of the intravascular member by means of a delivery tool may be performed simultaneously.

FIG. 32 is a perspective view of an arteriotomy needle, similar to the delivery needle of FIG. 31 but not including a delivery tool.

### DETAILED DESCRIPTION OF THE INVENTION

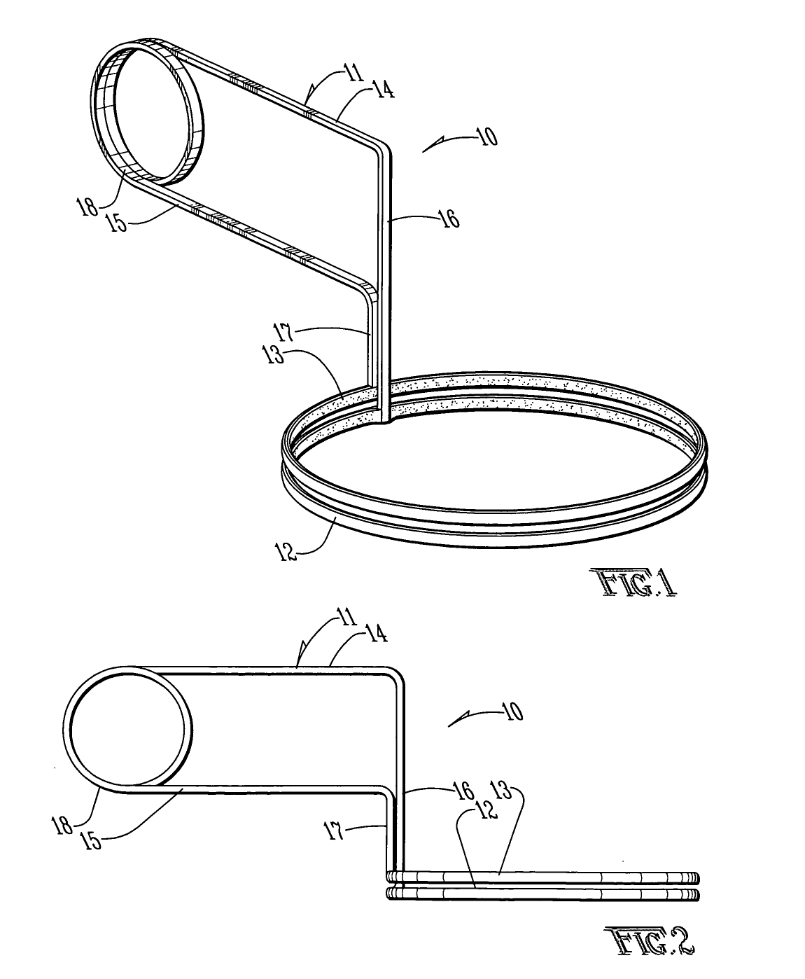

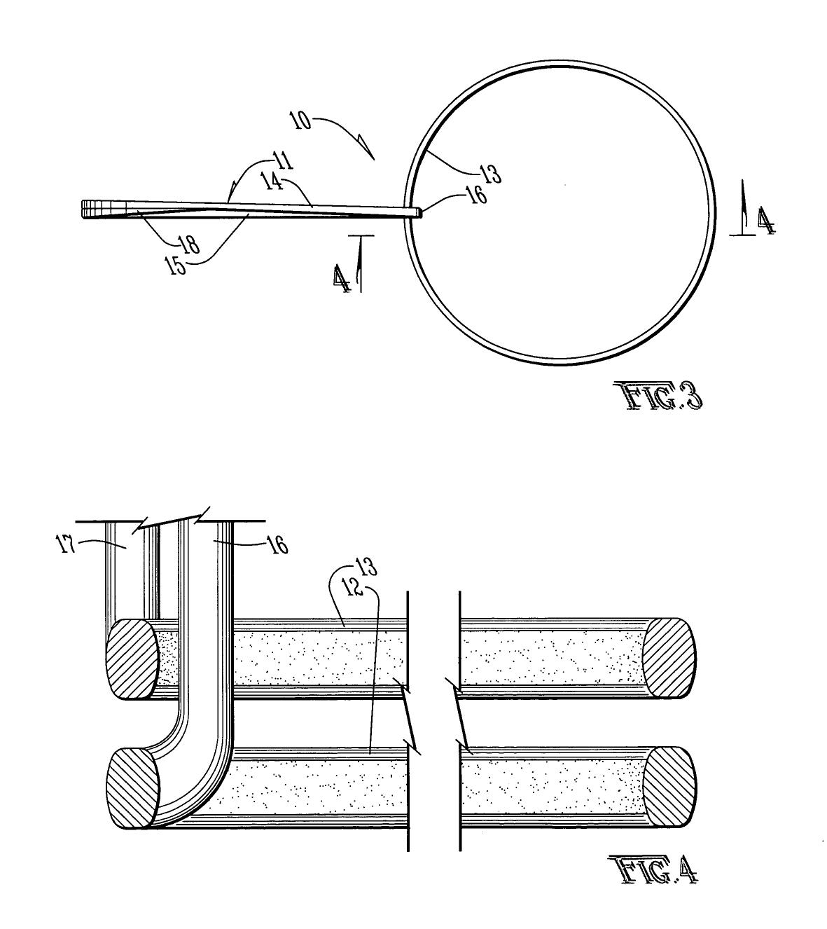

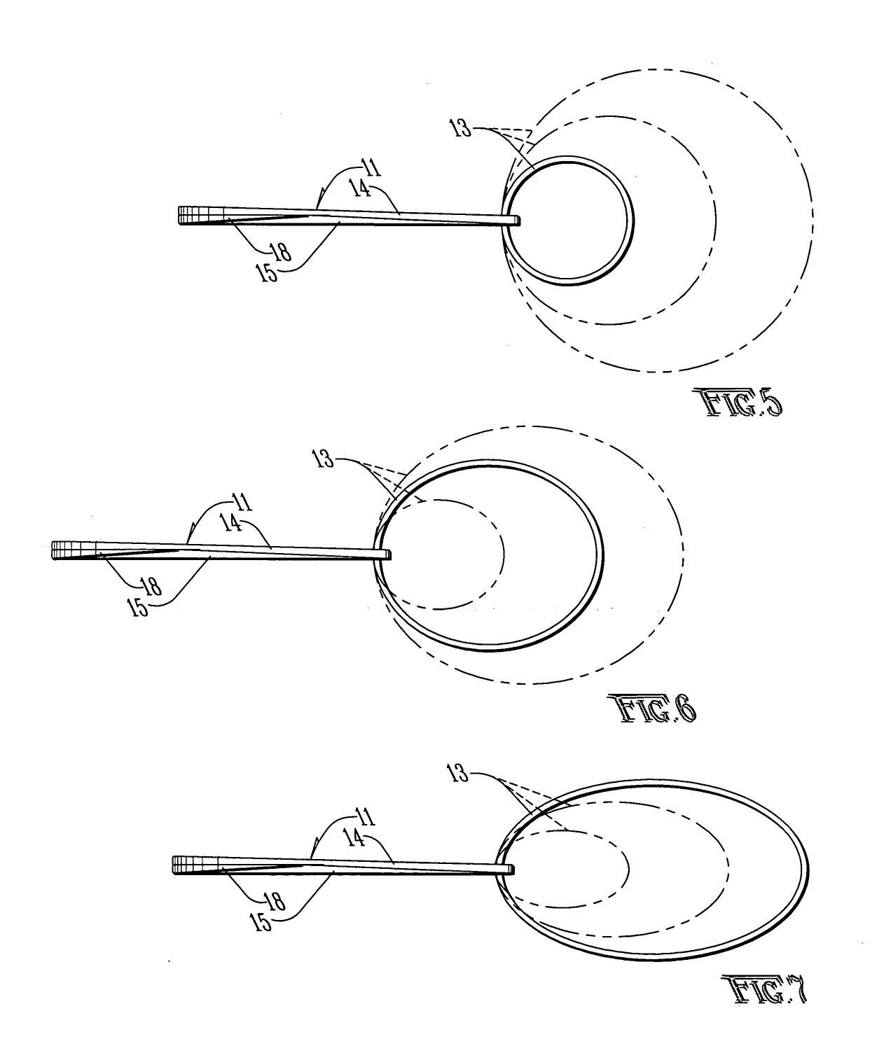

With reference to FIGS. 1-32, the preferred embodiments of the present invention may be described as follows.

The present invention is a clip for use in end-to-side anastomoses, i.e., in connecting the end of a graft vessel to the side of a recipient vessel in different parts of a human or animal body. As shown in FIGS. 1-4, the clip 10 comprises a handle 11 to which two rings, a lower ring 12 and an upper ring 13, are attached. The two rings 12, 13 are closely juxtaposed when the clip 10 is closed. The handle 11 may be similar to but smaller than the aneurysm clips known in the art, for example, the aneurysm clip disclosed in U.S. Pat. No. 4,765,335, the disclosure of which is incorporated herein in its entirety.

The clip 10 may be formed as shown in FIG. 1 from a single segment of material, such as by bending and forming a metal wire, or it may be formed in several pieces as will be described below. A preferred metal for constructing the clip 10 is titanium or any bio-compatible metal. It will be desirable in some applications that the material from which the clip 10 is made is compatible with medical imaging devices, such as MRI's. In surgical training applications where the clip 10 is used with a cadaver model, the clip 10 may be made of plastic or the like.

The handle 11 may additionally incorporate means to urge the two rings 12, 13 together by, for example, a resilient element such as a spring. The clip 10 may itself be made of a resilient material, such as an elastic metal, that may also incorporate a resilient element such as a coil spring. In the embodiment shown in FIG. 1, the handle 11 comprises a first arm 14 and a second arm 15. The first arm 14 is connected by a first offset segment 16 to the lower ring 12. The first offset segment 16 may in one embodiment be substantially perpendicular to said first arm 14 and to said lower ring 12. The second arm 15 is connected by a second offset segment 17 to the upper ring 13. The second offset segment 17 may in one embodiment be substantially perpendicular to said second arm 15 and to said upper ring 13. In other embodiments, the offset may be obtained by various combinations of straight and curved segments as desirable for various applications. Offsetting the main axis of the handle 11 from the plane containing the rings 12, 13 allows the lower ring 12 to be placed into an arteriotomy of a recipient vessel as described below while maintaining the handle 11 spaced apart from the recipient vessel for ease of manipulation of the handle 11. The two arms 14, 15 are connected by an integral coil spring 18 disposed so as to bias the two arms 14, 15 apart and therefore to bias the two rings 12, 13 together. However, by applying pressure urging the two arms 14, 15 together, the rings 12, 13 may be separated against the biasing force of the coil spring 18. The embodiment of FIGS. 1-4 may be formed integrally from a single segment of resilient material, such as a metal wire.

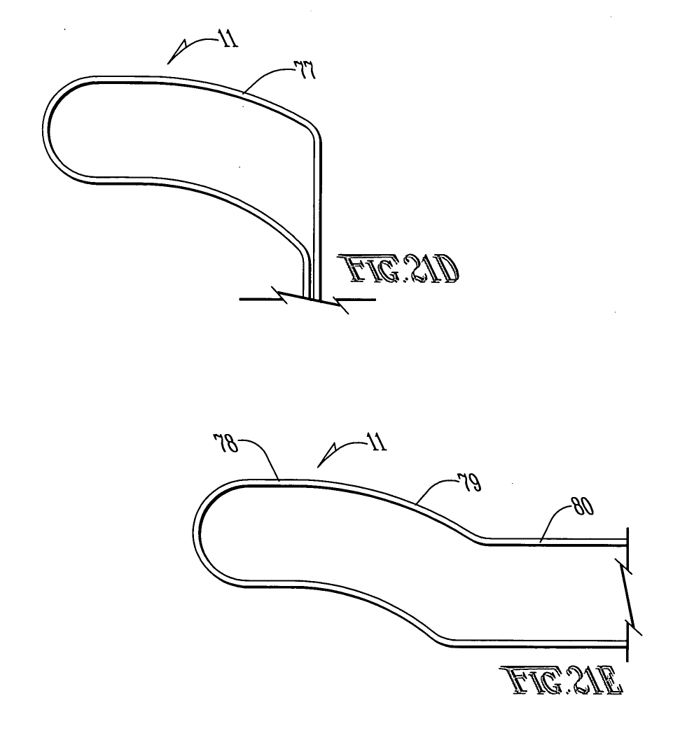

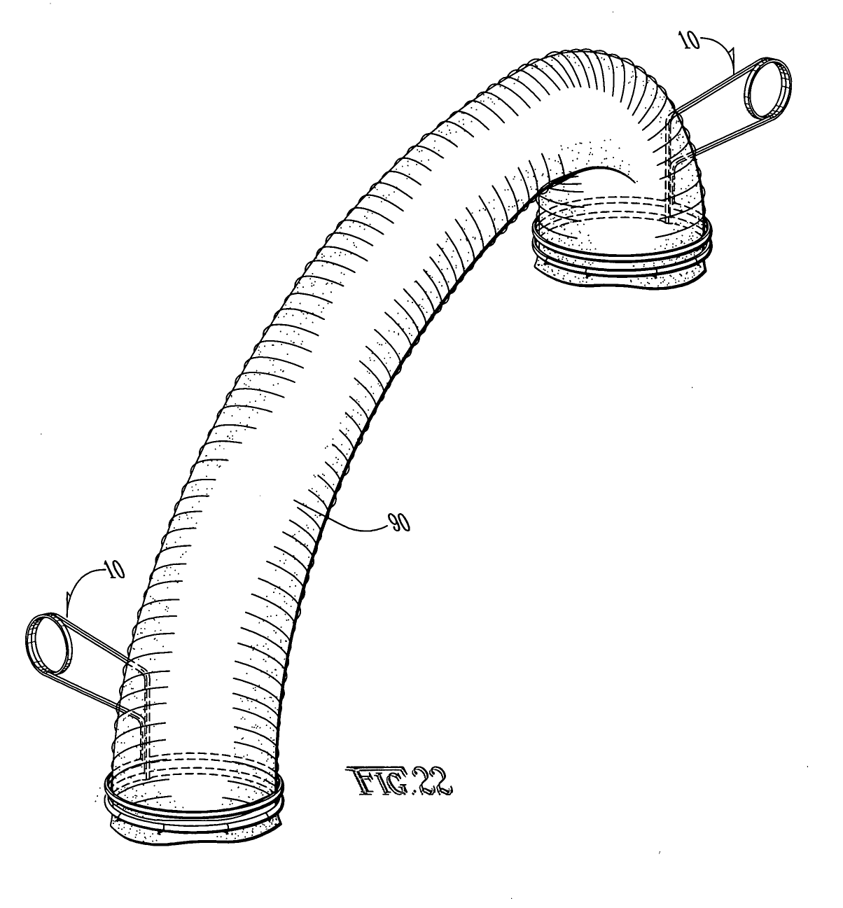

As shown in FIG. 18, the force to separate the two rings 12, 13 may be applied by a clip applier 20 to the arms 14, 15. (The figures only illustrate the tip of the clip applier and not the entire clip applier, which would be known to those of skill in the art.) The clip applier 20 may comprise a pair of members 21, 22 pivotally connected about a hinge joint 23. Each of said members 21, 22 comprises a jaw 25, 24 respectively, disposed anteriorly of said hinge joint 23 and a grip 26, 27 respectively, disposed posteriorly of said hinge joint 23 so that when the grips 26, 27 are manually closed, the jaws 25, 24 may be positioned to close onto the handles 14, 15 to urge the rings 13, 12 apart. The jaws 25, 24 may be curved as shown in FIG. 18 or, as shown in FIG. 19, the jaws 25, 24 may optionally be provided with buttons 28 on the interior faces of the jaws 25, 24 to act as low friction pivot points between the jaws 25, 24 and the arms 31, 32.

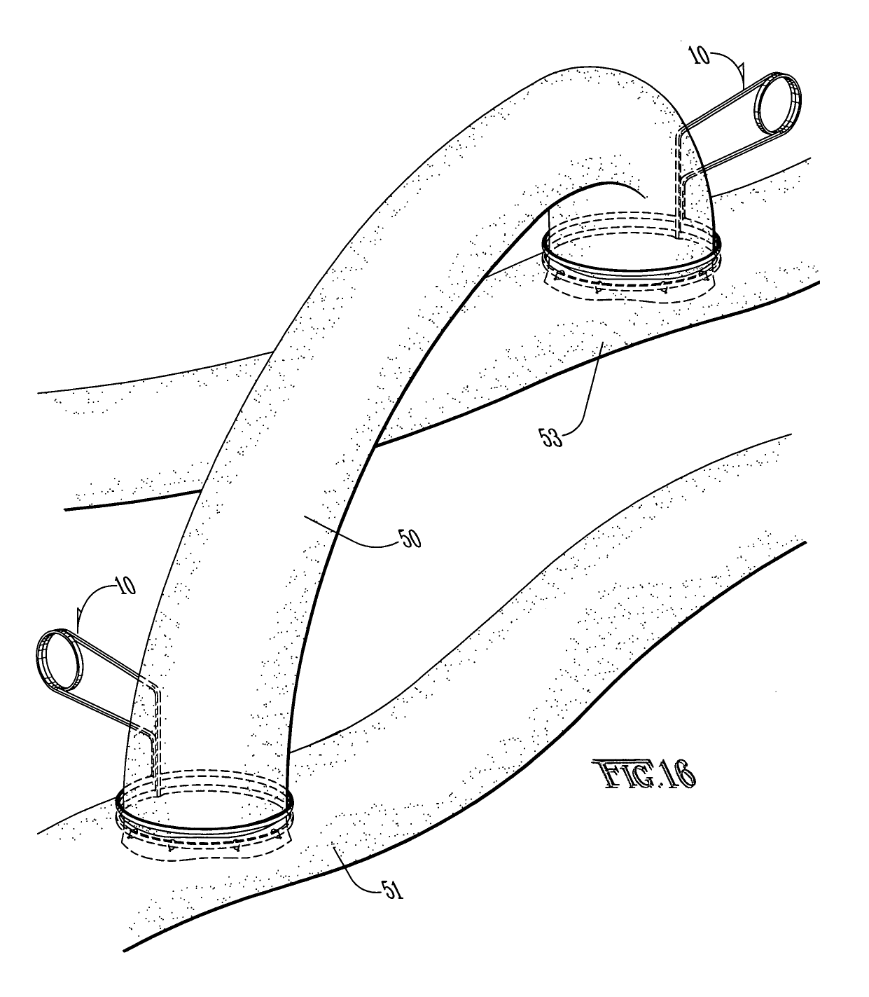

As illustrated in an alternative embodiment in FIG. 10, the clip 30 is made with two pivotal arms 31, 32 which are pivotally connected at a pivot point 33. The first pivotal arm 31 is connected to the first offset segment 16 and thence to the lower ring 12. The second pivotal arm 32 is connected to the second offset segment 17 and thence to the upper ring 13.

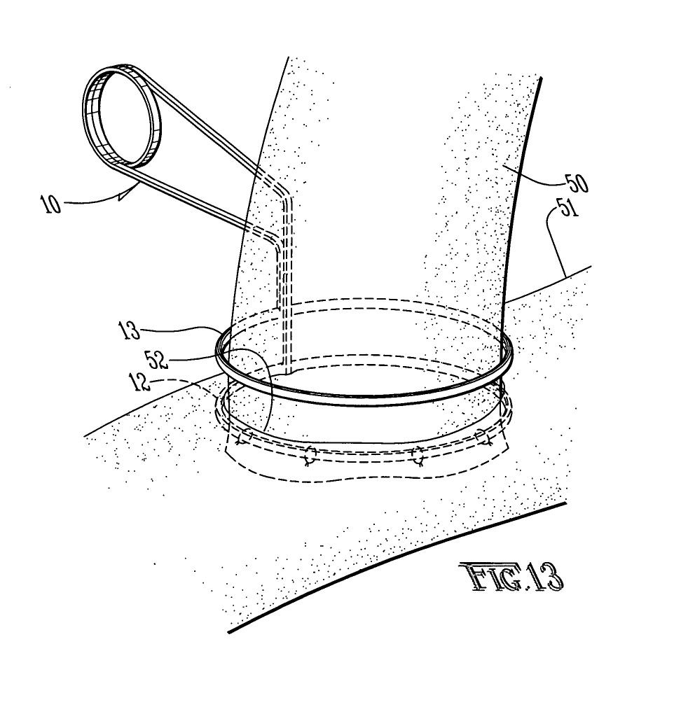

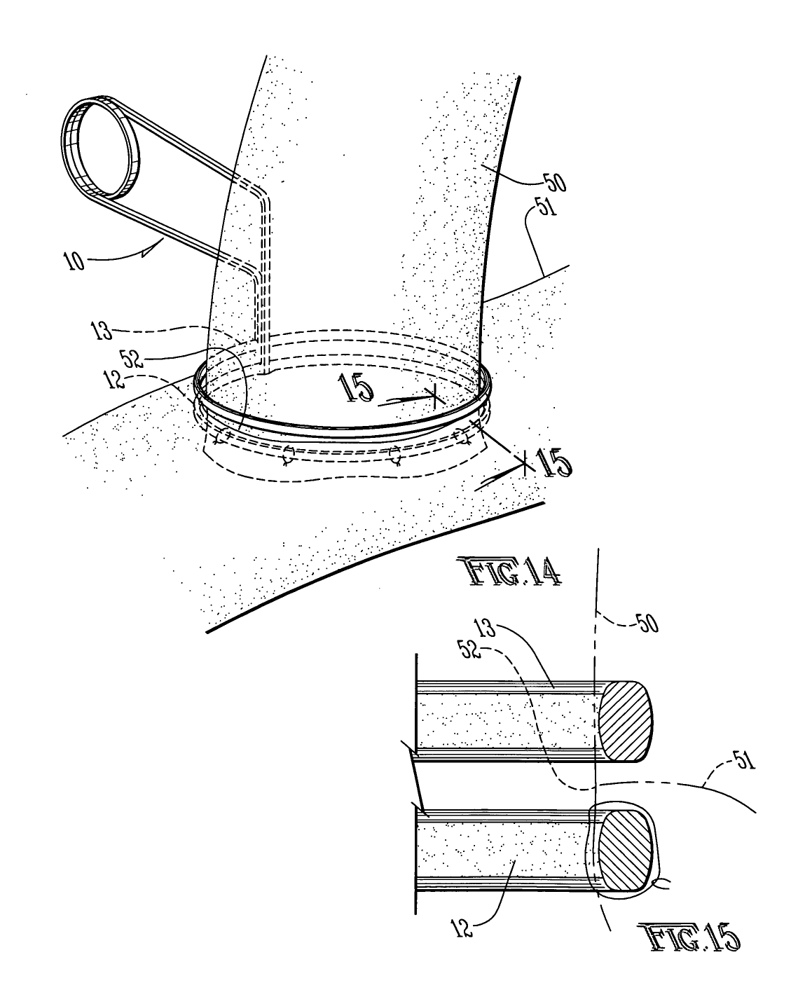

The resilient element that urges the upper ring 13 and the lower ring 12 together may have various forms other than the integral coil spring 18 shown in FIGS. 1-4. For example, FIGS. 8 and 10 illustrate an alternative embodiment in which the resilient element is a segment of resilient material 34 disposed between the arms 14, 15 or 31, 32. In the embodiments of FIGS. 9 and 11, the resilient element is a separate coil spring 35 disposed between the arms 31, 32 or 36, 37. In the embodiment of FIG. 9, the arms 36, 37 are connected together by a resilient arc 38. With the exception of the separate coil spring 35, the clip 40 of FIG. 9 may be formed integrally from a single segment of resilient material, such as a metal wire.

FIG. 11 illustrates an alternative embodiment having pivotally connected arms 31, 32 as shown in FIG. 10 and a separate coil spring 35 between the arms 31, 32 as shown in FIG. 9.

The handle 11 may be of different shapes depending on the various applications of the clip 10. For example, the handle 11 may include various combinations of straight and curved segments to accommodate various applications. For example, in FIG. 21A, the handle 11 includes a straight segment 71 disposed substantially parallel to the plane of the rings 13, 12 and operatively connected to the offset segments 16, 17 by an oblique downwardly angled straight segment 71. FIG. 21B shows an alternative embodiment of the handle 11 having a straight segment 73 disposed substantially perpendicular to the plane of the rings 13, 12 and connected first to a concavely curving segment 74 and thence to a straight segment 75 substantially parallel to the plane of the rings 13, 12. The straight segment 75 then connects to the offset segments 17, 16 (not shown) as described heretofore. FIG. 21C is an embodiment similar to that of FIG. 21A having an oblique straight segment 70, but with a more sharply downwardly angled segment 76. Oblique angles around 45° from the vertical are preferred. FIG. 21D shows a handle 11 that comprises a curved segment 77 that is curved along substantially its entire length. FIG. 21E shows a handle 11 that includes a substantially straight segment 78 that is disposed substantially parallel to the plane of the rings 12, 13 as heretofore described in reference to FIGS. 21A and 21C. The straight segment 78 is connected to a convexly curved segment 79 and thence to a second straight segment 80. The embodiments of FIGS. 21A-E are illustrative only and are not limiting to the full scope of the invention which may include various other shapes of handles to accommodate any applications for a vascular clip as would be apparent to one of skill in the art.

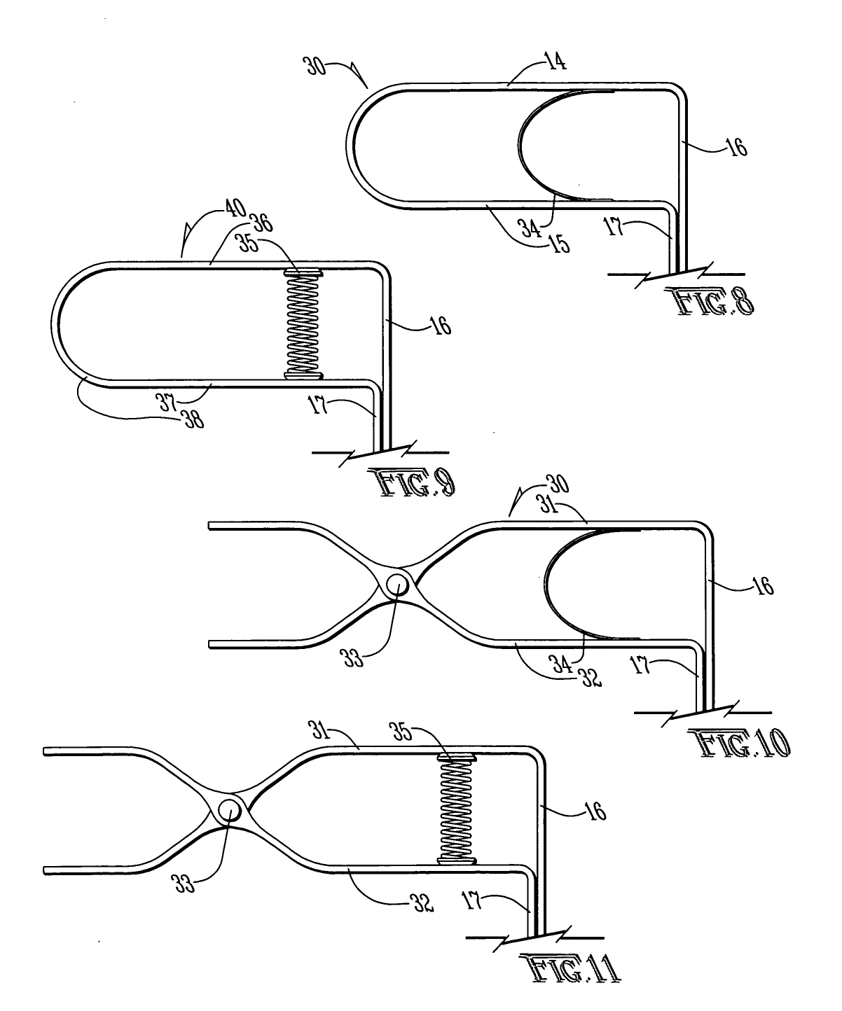

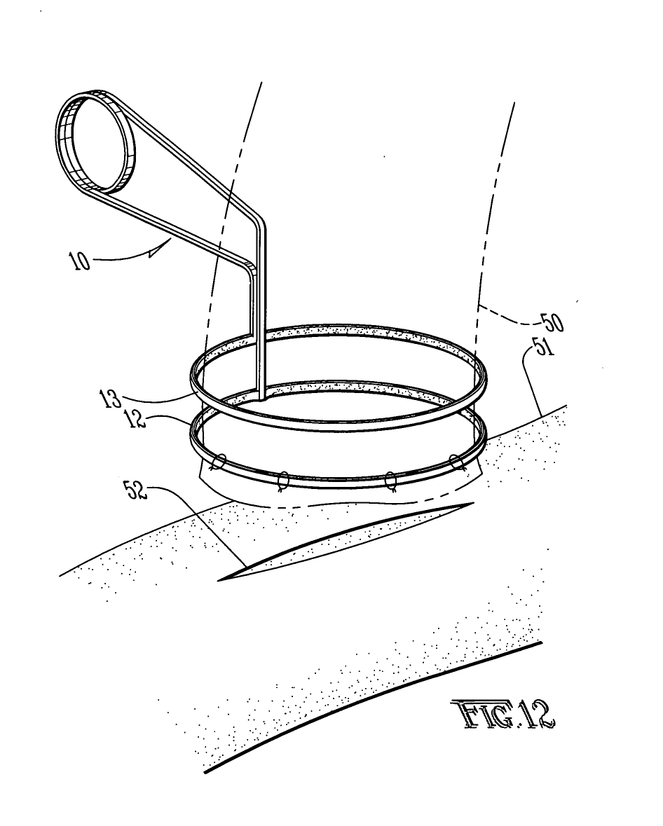

As shown in FIGS. 5-7, the rings 13, 12 may also have different shapes and sizes to accommodate different kinds of anastomoses and different vessels of all sizes in all parts and cavities of the human or animal body, including without limitation the brain, thorax, abdomen, pelvis, neck and limbs. FIG. 5 illustrates rings 13 that are substantially circular in varying sizes from large to small. Likewise, FIG. 6 illustrates rings 13 with an broad elliptical shape and FIG. 7 illustrates rings 13 with narrower elliptical shape. Although the lower ring 12 is not shown in FIG. 5-7, the two rings 12, 13 are preferably substantially the same shape and size. The rings 12, 13 may have a maximum dimension (for example, a diameter in the case of a circular shape or a major axis in the case of an elliptical shape), which preferably is in the range of from around 2 mm up to around 2.5 cm, although the present invention is not limited to this size range. When the clip is used with small vessels, for example in cerebrovascular anastomoses within the skull, it is desirable that the size of the lower ring 12 and the handle 11 be as thin as possible.

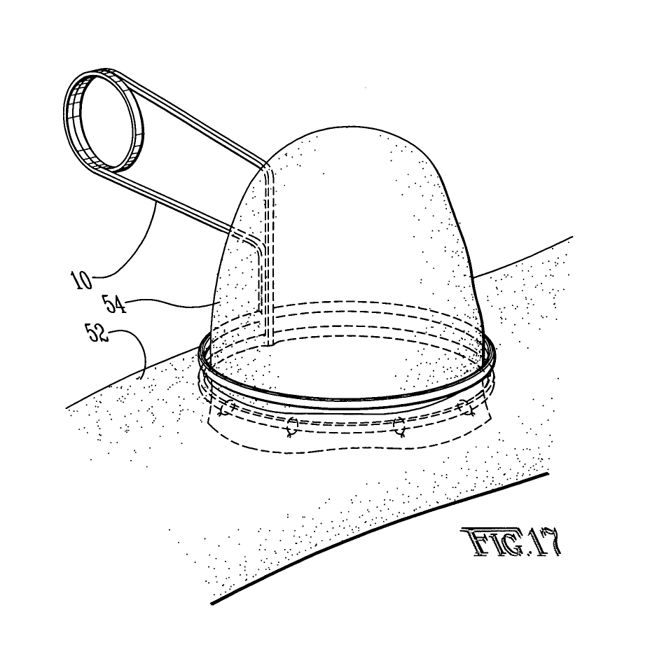

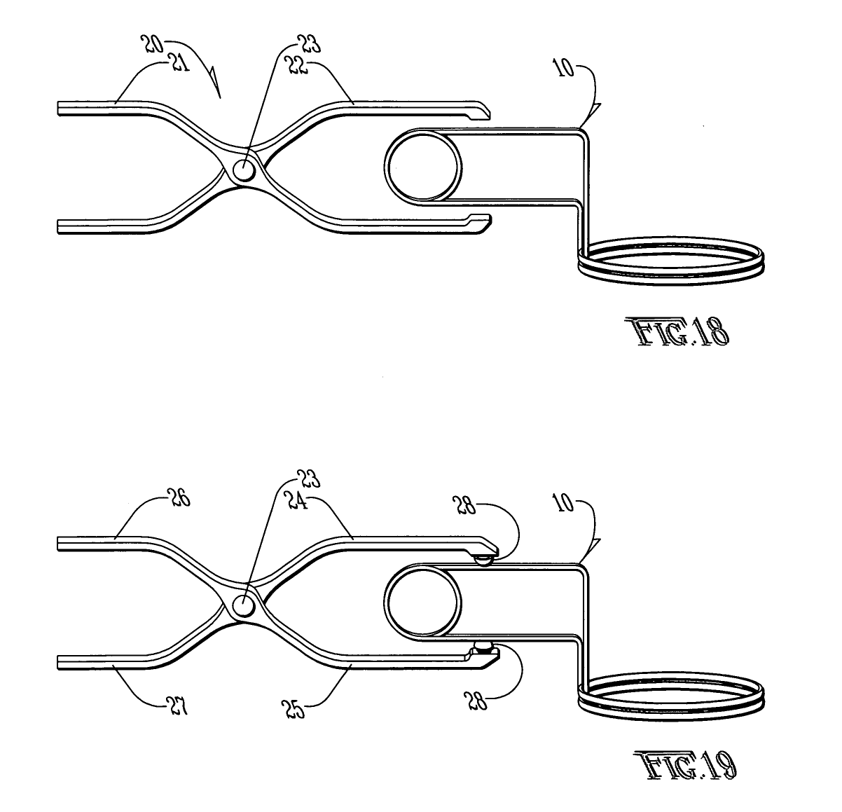

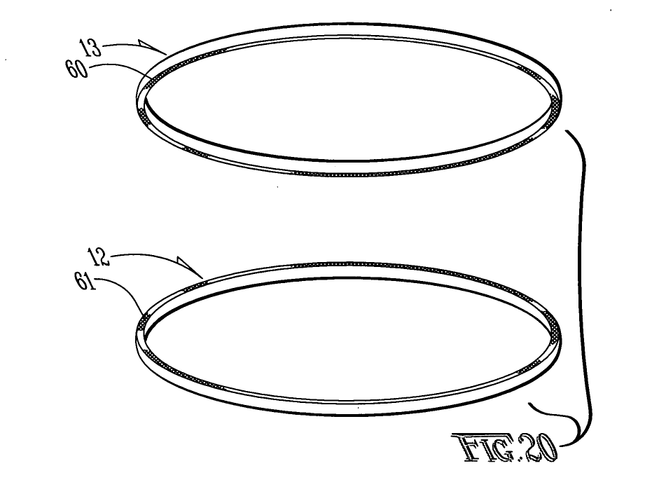

As shown in FIGS. 12-15, the clip 10 may be used to form an anastomosis by attaching a graft vessel 50 to a recipient vessel 51. As shown in FIG. 12, one end of the graft vessel 50 is inserted inside the two rings 12, 13 and an edge of the open end of the graft vessel 50 is sutured to the lower ring 12 while the clip 10 is held open to allow such suturing. A hole 52 (called an “arteriotomy”) is made in the recipient vessel 51 that is smaller than the diameter of the rings 12, 13. As shown in FIG. 13, the lower ring 12 holding the open end of the graft vessel 50 is inserted into the hole 52 in the recipient vessel 51 while the clip 10 is held open so that the edge of the hole 52 is positioned between the two rings 12, 13. A shown in FIGS. 14 and 15, the clip 10 is then closed to catch the edge of the hole 52 in the recipient vessel 51 between the two rings 12, 13 as they are closed tightly and the anastomosis is thereby completed. The graft vessel 50 can be a vein or an artery (veins are preferred as having less thickness), either autologous or heterologous, or the graft vessel 50 can be made of a synthetic material. In the later case the graft vessel 50 may be attached to the lower ring 12 during the process of manufacturing the clip 10.

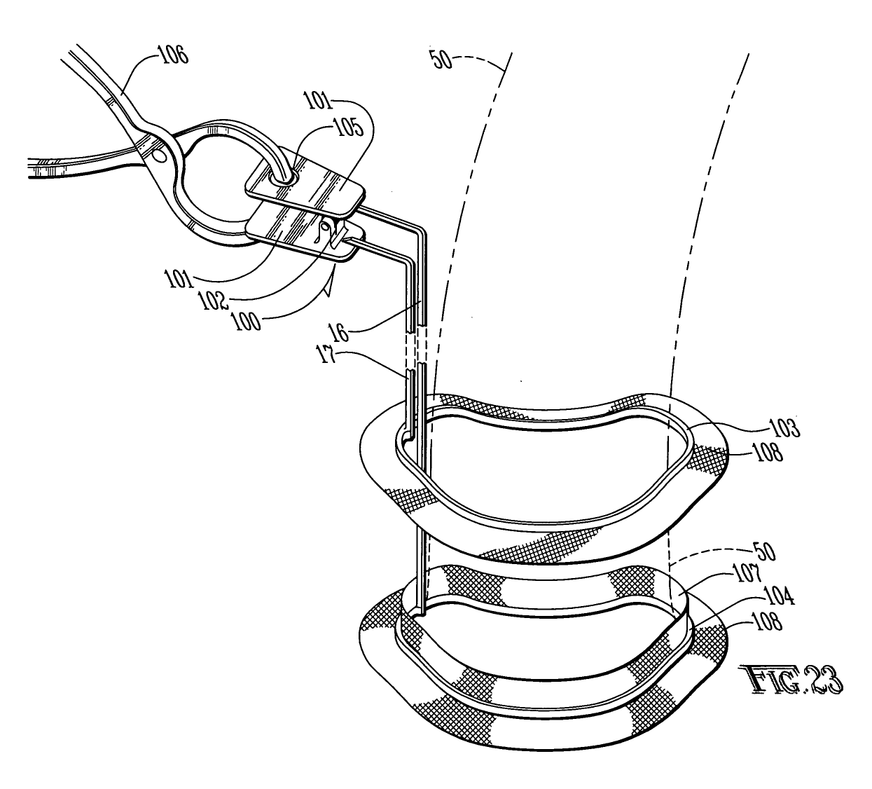

To ensure a firm grasp of the rings 12, 13 on the walls of the recipient vessel 52, the lower surface of the upper ring 13 and the upper surface of the lower ring 12, i.e., the surface of each ring 12, 13 facing the other ring, may be provided with a rough surface 60, 61 as shown in FIG. 20. The surface may be roughened by any of various means known in the art, including parallel or crosshatched grooves. Alternatively, the inner surface of one ring may be provided with spines (not shown) that fit into complementary holes (not shown) on the facing surface of the other rings.

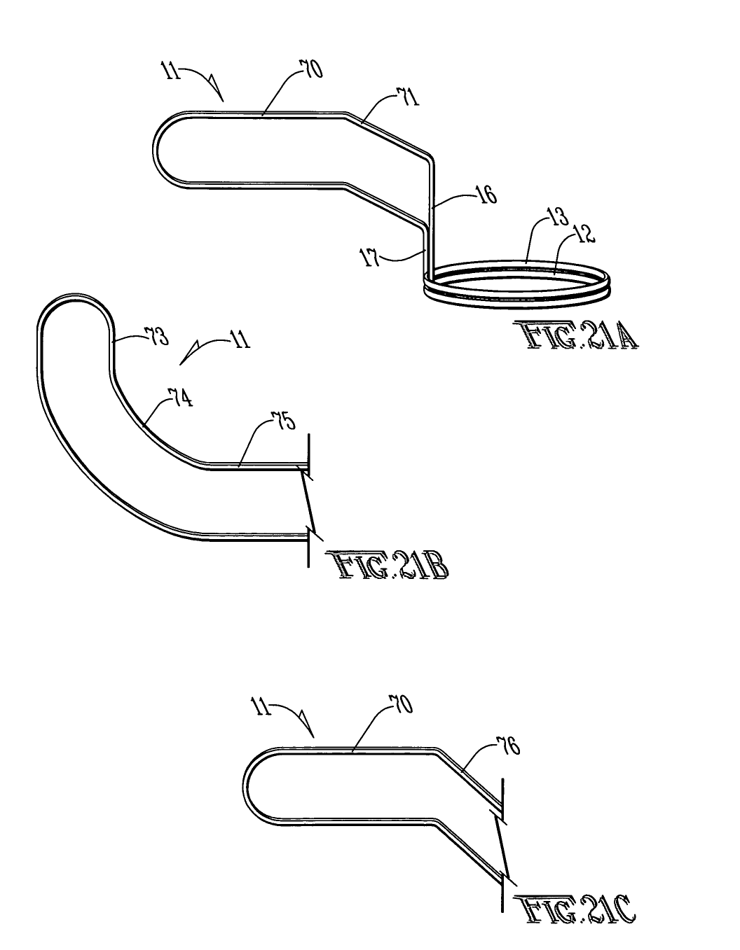

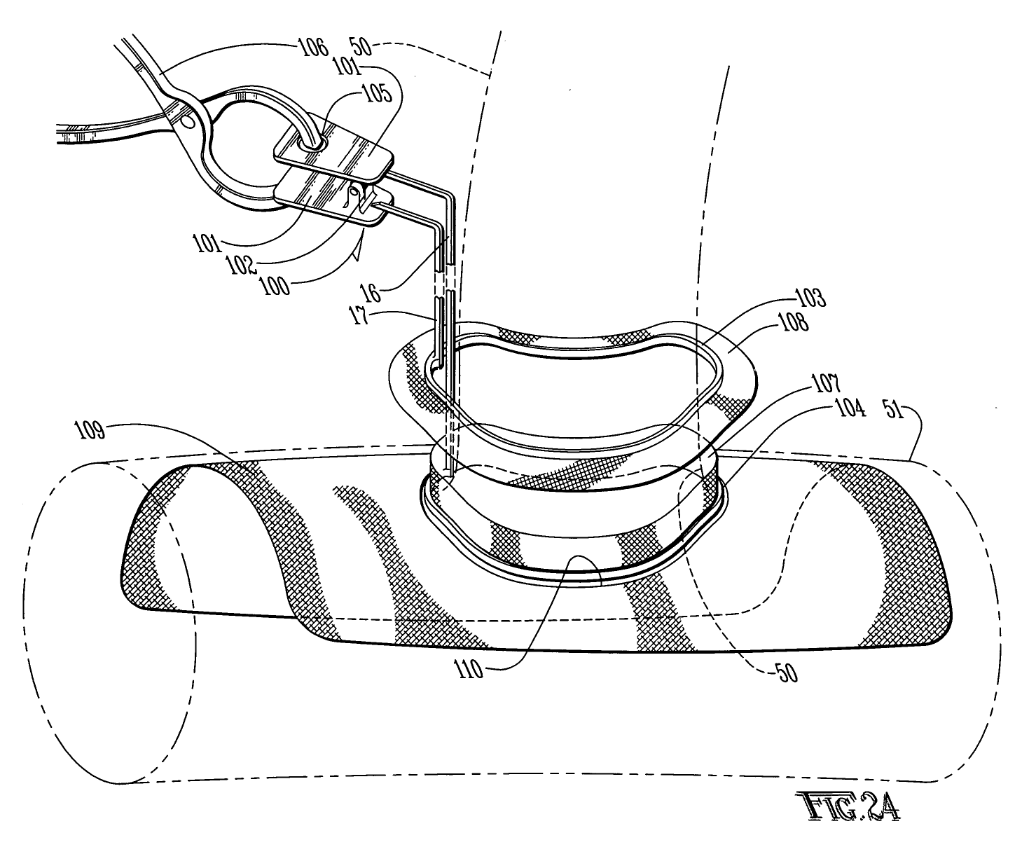

With reference to FIG. 16, a clip 10 may be attached as described above to each end of a graft vessel 50 so that the graft vessel 50 may be connected between a donor vessel 53 and a recipient vessel 52. FIG. 22 shows an alternative version with a corrugated graft 90 that may be stretched to a desirable length between a donor vessel 53 and a recipient vessel 52. This avoids placing any force on the graft between the donor and recipient vessels and allows various maneuvers while applying and attaching the clips to the vessels. Such a corrugated form is well known from vascular grafts made of TEFLON for major vessels such as the aorta. The graft 50, 90 may be made in different sizes, including different diameters corresponding to the sizes and diameters of the rings 12, 13. The graft 50, 90 may also be made in different lengths.

In an alternative embodiment illustrated in FIG. 17, instead of a graft vessel 50, an artificial aneurysm 54 may be attached to the lower ring 12 of the clip 10 as described above so that the artificial aneurysm 54 may be connected to a recipient vessel 52. This embodiment may be particularly useful in surgical training and may be used in conjunction with a training model such as the cadaver model described in U.S. Pat. No. 6,790,043 to the same inventor as the present invention. The artificial aneurysm 54 may be applied in the teaching of different treatment modalities, such as coiling and endovascular treatment.

As described above, certain embodiments of the present invention comprise an extravascular member, such as an upper ring, an intravascular member, such as a lower ring, and means to fix the intravascular member in the recipient vessel, such as a handle with a resilient element biasing the extravascular member and the intravascular member together. A graft vessel is attached to the intravascular member around an edge of an opening in the intravascular member. When means other than a biasing resilient element are employed to fix the intravascular member in the recipient vessel, certain embodiments of the present invention may not require an extravascular member. In other embodiments of the present invention, means may be employed to insert the intravascular member into the recipient vessel, for example, delivery systems that open an arteriotomy while simultaneously delivering the intravascular member into the recipient vessel. These and other alternative embodiments are described below.

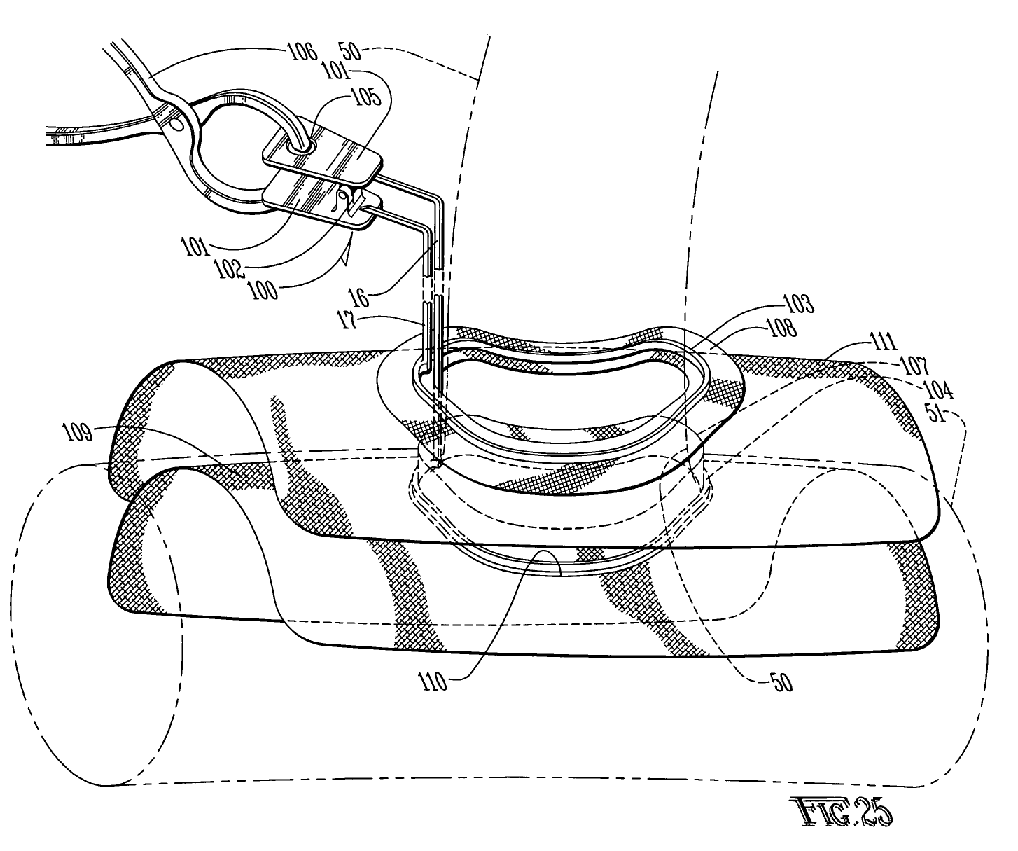

FIG. 23 shows a further embodiment of clip 100 where the handle has two plates 101 biased apart by a resilient element, for example, by a coil spring 102. The resilient element may be located as shown in FIG. 23 toward the anterior ends of the plates 101 or may be located toward the posterior ends of the plates 101. The plates 101 are attached to upper and lower rings 103, 104 through the resilient element and the handle as described more fully in the discussions above.

As also shown in FIG. 23 the plates 101 may have an indentation 105, for example, a rounded depression, where the clip applier 106 can catch the handle plates 101. The indentation 105 associated with one of the plates 101 is shown in FIG. 23. A similar indentation 105 associated with the other plate 101 is not shown. The clip applier 106 may also have a complementary shape to the indentation, for example a hemisphere to fit into a rounded depression, similar to the buttons 28 described above.

FIG. 23 also shows that the rings 103, 104 may have outwardly extending lips 108 to improve the grasp of the rings 103, 104 on the recipient vessel 51 by engaging more of the surface of the vessel walls and the lower ring 104 may also have an upwardly extending lip 107 to suture to the graft 50. The lips 107, 108 may be formed from an extruded or flattened wire or could be in the form of material added to a wire. The lips 108 help to form the anastomosis when the arteriotomy 52 is not uniform. The lips 107 may be made of a suturable material or if the lip 107 is made from metal, it may be provided with holes (not shown) for suturing. The lips 107, 108 may be made in various shapes and sizes. For example, the lips 107, 108 may be relatively small as shown in FIG. 23 or relatively large as shown in FIG. 24. The lips 107, 108 may also have various shapes and the present invention is not limited to the shapes illustrated and described herein. The rings 103, 104 may be shaped to conform to the curvature of the recipient vessel 51 to improve the grasp of the rings 103, 104 on the walls of the vessel and to prevent kinking of the vessel. As described above, a graft vessel 50 is attached to the intravascular member, for example, in the case of a lower ring 104, at its end around the edge of the lower ring 104. The device may be manufactured with artificial grafts made from synthetic or other materials known in the art built into the intravascular member, for example the lower ring 104, at the time of manufacture. Furthermore, autologous or heterologous grafts (arterial or venous) can be attached to the intravascular member. In the embodiment of the intravascular member where it is a ring with an upwardly extending lip 107 made of suturable material, the graft vessel 50 can be attached by suturing. To use the device of FIG. 23, the lower ring 104 is placed inside the arteriotomy 52 and fastened as described above.

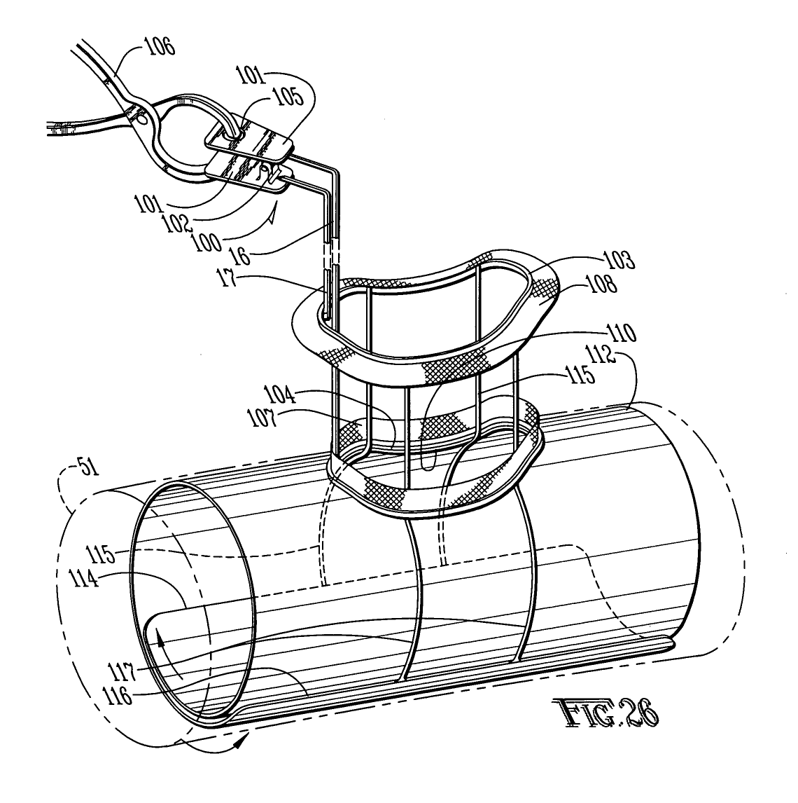

FIGS. 24 and 25 show an alternative embodiment of the device of FIG. 23 where the intravascular member is a half tubular or a hemi-cylindrical body 109 with an opening 110. The graft vessel 50 is attached to an edge around the opening 110. The opening 110 may incorporate a ring for attachment of the body 109 or the ring may have an upwardly extending lip as described above for ease of attachment of the graft vessel 50. The upwardly extending lip may be shaped to conform to the shape of the body 109. The body 109 may be made of mesh or part mesh as described below and may be of various sizes for accommodating various applications. This embodiment of the intravascular member may be employed with larger arteriotomies or other applications. The extravascular member may be an upper ring 103 as shown in FIG. 24 or may also be a half tubular or body hemi-cylindrical body 111 that is identical to the intravascular member described with respect to FIG. 24. The body 111 has an opening through which the graft vessel 51 passes. This embodiment may be used where the recipient vessel 51 is straight and it is desirable to keep the vessel open. It is desirable that the corners of the bodies 109, 111 be contoured or rounded to allow for easier insertion into an arteriotomy.

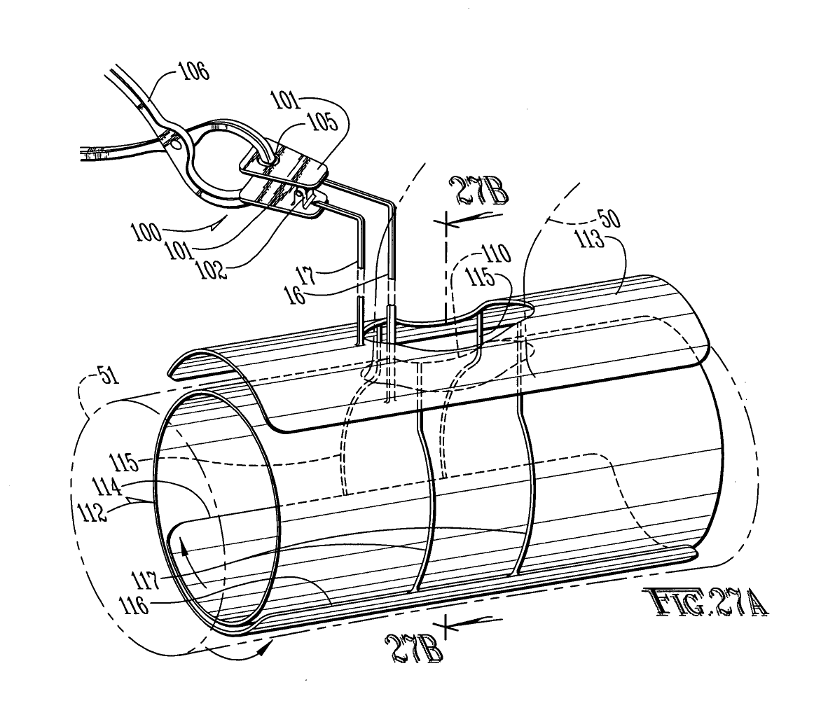

In other alternative embodiments, the intravascular member (the member that is inserted into the recipient vessel) may be an expandable cylinder as shown in FIGS. 26-29B.

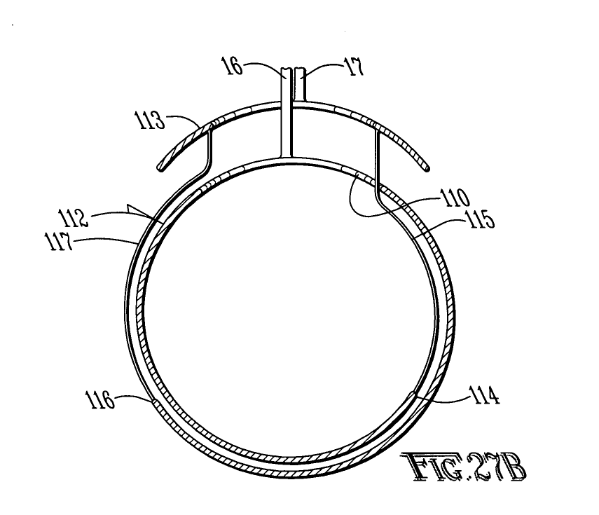

FIGS. 26-27B shows an embodiment of the device where the expandable cylinder is a scroll-like body 112. The body 112 may have shape memory properties or resilience allowing it to expand when the handle is released and to thereby fix itself against the inner surfaces of the wall of the recipient vessel 51. The extravascular member may be a ring 103 as shown in FIG. 26 or may be a half tubular body 113 as shown in FIG. 27A that is identical to the body 111 described above with respect to FIG. 25. In either case, a graft vessel 50 is attached to an opening in the intravascular member, in this case the scroll-like body 112. With reference to FIGS. 27A and 27B, the scroll-like body 112 has an inner edge 114 that is attached by one or more flexible wires 115 passing through the interior of the body 112 to an opening in the mesh of the body 112 and thence around the exterior of the body 112 to the opening 110 to the extravascular member, such as the hemi-cylinder 113. The scroll-like body 112 has an outer edge 116 that is attached by one or more flexible wires 117 around the exterior of the scroll-like body 112 to the extravascular member, such as the hemi-cylinder 113. It is desirable that the wires 115, 117 be attached to the extravascular member at the innermost edge of the opening in the extravascular member. The offset segment 16 is attached to the scroll-like body 112. The offset segment 17 is attached to the hemi-cylinder 113. When the plates 101 are compressed by the clip applier 20, the extravascular member, such as the hemi-cylinder 113, is forced away from the scroll-like body 112 and the wires 115, 117 pull the edges 114, 116 so as to roll up and compress the scroll-like body 112. After the body 112 is placed into the arteriotomy 52, the clip applier 20 is removed, the biasing spring 102 closes the extravascular member onto the recipient vessel 51 as the scroll-like body 112 relaxes and expands inside the recipient vessel 51 so as to fix the device in place. FIG. 26 is an alternative of the embodiment in FIGS. 27A and 27B in which the extravascular member is a ring. In either embodiment, other means known to those skilled in the art could be used to compress the scroll-like body 112.

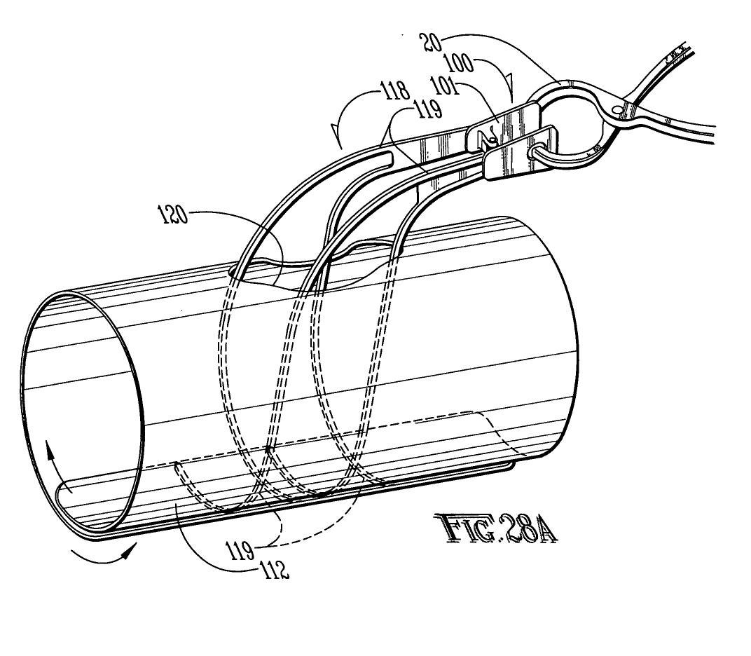

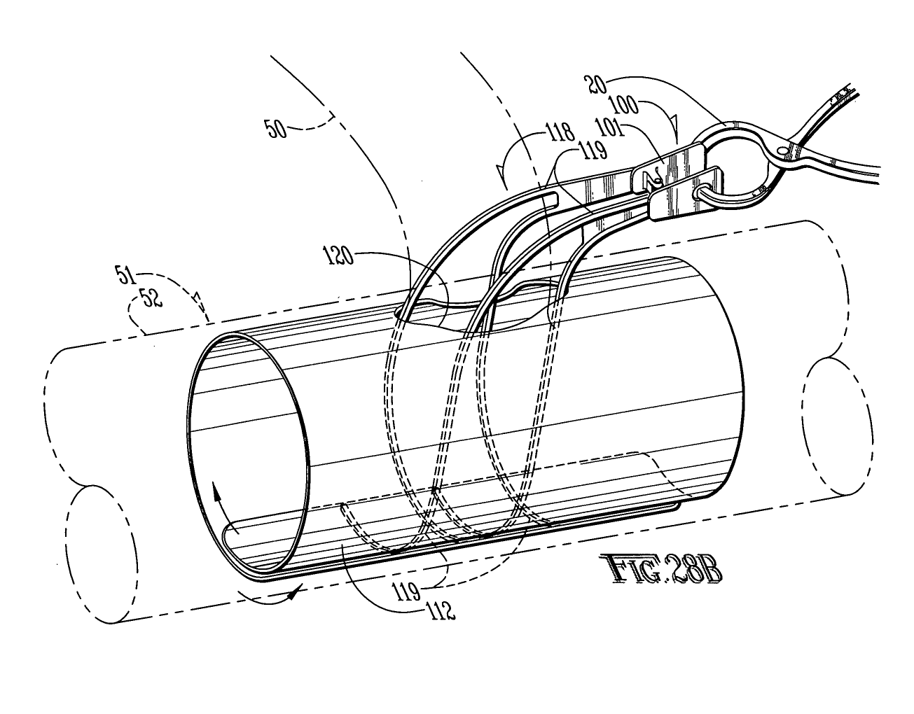

FIGS. 28A and 28B show a further embodiment of the device where the scroll-like body 112 is attached to the handle 118. The handle 118 is attached to the exterior of the scroll-like body 112, by means of a pair of arms 119, one arm 119 to either side so that compression of the handle 118 compresses the body 112. The arms 119 are smoothly curved for ease of insertion into an arteriotomy. The scroll-like body 112 has a round or oval opening 120 in its middle, or at the junction of the middle third with the terminal third of the upper surface (the surface facing the handle 118). This opening 120 is attached to graft vessel 50 as previously described. The body 112 may be solid, a mesh covered with a membrane or partly-meshed/partly-solid where the lower part of the body 112 is meshed. The body 112 is compressible due to the scroll-like shape as described above. When the two plates 101 of the handle 118 are compressed by the clip applier 20, the body 112 is in its compressed state (smallest size and diameter) which allows it to be inserted into a small arteriotomy 52 the wall of the recipient vessel 51. This embodiment acts like a stent and is useful if there has been injury to the recipient vessel 51. To use this embodiment the physician holds the handle 118 with the clip applier 20 compressing the two plates 101 together and inserts the body 112 in the arteriotomy 52 of the recipient vessel 51. The handle 118 compresses the exterior of the body 112. When the handle plates 101 are released, the body 112 inside the recipient vessel 51 expands due to its resilient character or due to being made of shape memory materials. The body 112 is fixed by compressing the walls of the recipient vessel 51 from inside. This embodiment can be delivered without a handle 118 by using a delivery needle as described below or by using a readily available microforceps to compress the body 112 and deliver it into the recipient vessel 51.

The embodiments where the intravascular member is an expandable cylinder may also include a stent-like device made of a deformable mesh material.

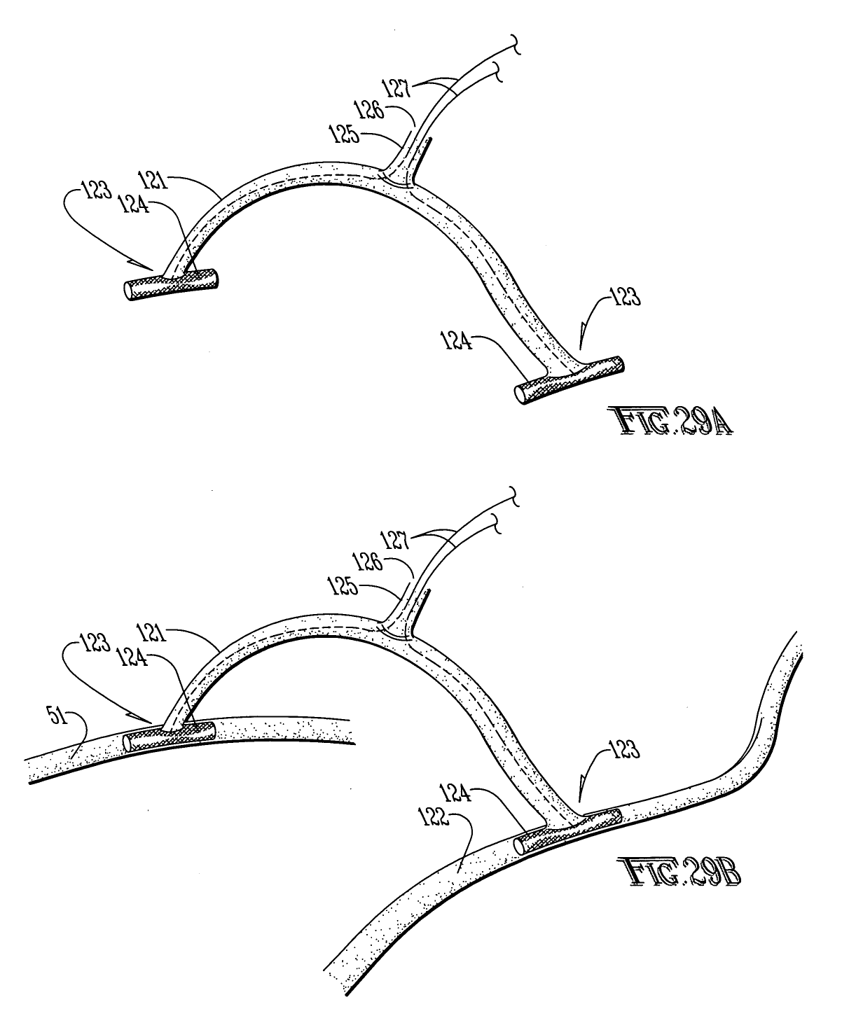

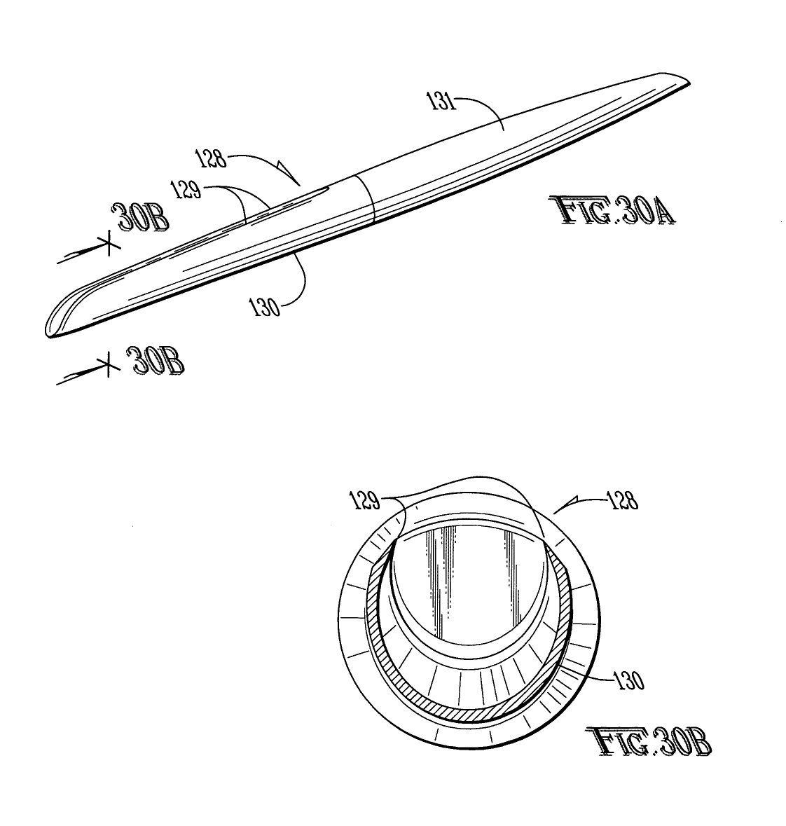

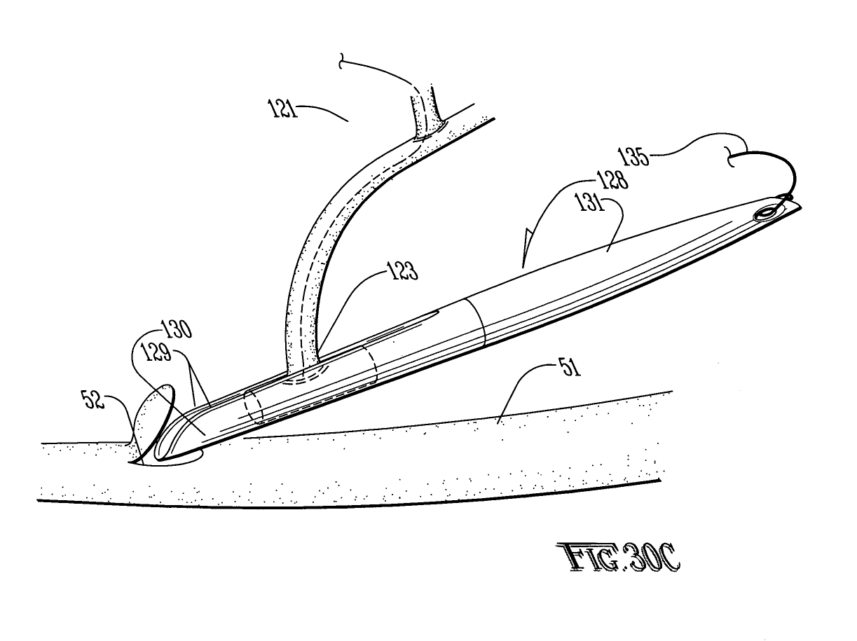

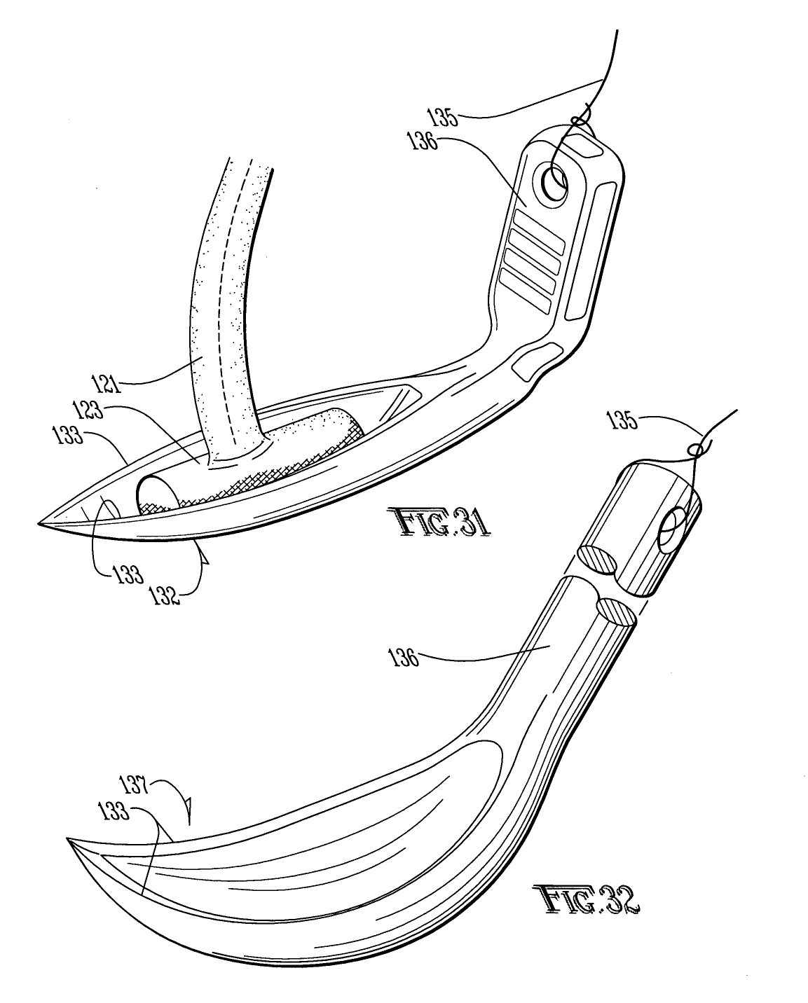

FIGS. 29A and 29B show a further embodiment of the device in which a graft vessel 121 is connected to both a recipient vessel 51 and a donor vessel 122 by means of T-shaped ends 123 having horizontal short shafts holding a stent-like intravascular member 124 built-in and embedded in the walls of the graft vessel 121. By using the term “T-shaped,” it is not meant to imply that the ends are necessarily at right angles. Angles other than right angles are also contemplated as being within the scope of the present invention. Arteriotomies 52 may first be made in the recipient and donor vessels 51, 122 and the T-shaped ends 123 inserted into the arteriotomies 52 using a guide wire as is known in the art. A delivery needle as described below may be used to make the arteriotomies 52 and insert the T-shaped ends 123 in a single step. The graft vessel has in its middle portion a side branch 125 with an opening 126 that may be closed with a hemostat clip. Two T-shaped balloon catheters 127 along with guide wires (not shown) pass through the opening 126 into the branch and thence each catheter 127 proceeds in opposite directions within the graft vessel 121 to respective T-shaped ends 123, where the balloon (not shown) of the catheter 127 is placed inside the respective stent-like intravascular member. The balloons of the catheters 127 in initially deflated so the stent-like intravascular member 124 is in its smallest size and diameter. The tubes of the balloon catheters 127 exit the branch 125 of the graft vessel 121 along with the guide wires with enough length to allow inflation and deflation of the balloons. This embodiment with an artificial graft may be manufactured in one piece, including the balloon catheters and inflation tubes with guide wires. To use this embodiment of the graft vessel 121, the T-shaped ends are inserted in the recipient and donors vessels 51, 122, respectively, by using, for example, the delivery needle described below with respect to FIGS. 30C and 31. The balloons of the catheters 127 after pulling out the guide wires are then inflated so that the stent-like intravascular member 124 is expanded and is fixed by compressing the walls of the recipient and donors vessels 51, 122. The balloons are then deflated and pulled out of the graft vessel 121. The opening of the branch 125 is closed using a hemostat clip.

FIGS. 30A-30C show a delivery needle 128 having two sharp edges 129 that serve to open the recipient vessel 51 with a precise arteriotomy 52 while delivering the intravascular member inside the recipient vessel 51. This allows the arteriotomy to be made and the intravascular member inserted without occluding the recipient vessel 51.

To make the arteriotomy 52 in the recipient vessel 51, the needle 128 as shown in FIG. 30B has a boat-like cross-sectional shape with sharp upper cutting edges 129 on the anterior portion 130 of the needle 128 so that when the needle 128 is inserted in the recipient vessel 51 it cuts the wall of the vessel and lift up a flap of the vessel wall. The anterior portion 130 of the needle 128 is provided with sharp cutting edges 129, but the posterior portion 131 is blunt. The intravascular member, for example the T-shaped end 123 of FIGS. 29A and 29B, fits into the posterior portion 131 and may be pushed off the needle 128 and into the arteriotomy 52 once it is formed as shown in FIG. 30C. A string 135 may be attached to the end of the needle 128 to aid in retaining and removing the needle 128 so as to avoid loosing the needle 128 in the arteriotomy 52. The needle 128 may be inserted by holding the end of the needle in a pair of forceps. An alternative embodiment of the delivery needle 132 is shown in FIG. 31. The delivery needle 132 has a body with a boat-like cross sectional shape and sharp upper cutting edges 133 to make an arteriotomy 52 when inserted into the recipient vessel 51. The needle 132 has a hollow interior portion 134 that may receive the intravascular member so that the arteriotomy 52 and the delivery of the intravascular member may be performed simultaneously. As with the embodiment above, a string 135 may be attached to the end of the delivery needle 132 to avoid the possibility of loosing the needle in the arteriotomy 52. Once the needle 132 has made the arteriotomy 52, the intravascular member is pushed into the arteriotomy 52 using a micro-forceps or an appropriate delivery tool. FIG. 32 is a perspective view of an arteriotomy needle 137 which includes a string 135. The needle 137 only makes the arteriotomy and the intravascular member is delivered by other means. The needles 132, 137 may include handles 136 extending upwardly from the posterior portion of the needles 132, 137. The handle 136 may be of various lengths for ease of grasping by various delivery tools. It is desirable that the handle be flattened in a plane transverse to the longitudinal axis of the needle or cylindrical in shape to allow for it to be held by a needle holder of the type known to those skilled in the art.

The device of the present invention may be made from stainless steel, shape memory materials and other materials known in the art that serve the purpose and function of the device or parts of the device. It is desirable that all artificial parts in the device that will be in contact with the blood stream are coated with biogenic materials or other materials (known in the art) that help in preventing thrombosis formation or being the source of embolisms; for example, the materials may be coated with human cells.

The prior art of anastomoses are much more complicated than the present invention, are composed of several pieces, and take longer to perform the procedure and are more expensive. The present invention is much simpler with a wide range of uses and applications. Non-occlusive anastomoses, for example, those using lasers, are considered highly desirable in that the patient is at less risk due to the minimizing the period of time that a vessel is occluded. An occlusion time of no more than 3 to 5 minutes is considered non-occlusive and the present invention can achieve this limited period of occlusion.

Using the clip of the present invention will shorten surgery time and minimize occlusion time during regular anastomoses. The different sizes and shapes of the clip with artificial grafts and other devices described above provide a wide range of uses. In addition, the various embodiments of the clip with an artificial graft vessel will provide the vascular surgeon with a wide range of choices in vascular surgery. The delivery needle may be employed in various kinds of anastomoses and bypasses. The present invention can be used on vessels of 2 mm to the largest vessels in the body.

The current preference in surgery is for minimally invasive surgery. The present invention allows different types of anastomoses to be done in a minimally invasive way and so shorten the time of surgery. Most importantly, it will shorten the occlusion time of the arteries which can lead to stroke.

The present invention has been described above with reference to vascular anastomoses. However, the invention is not limited to anastomoses with the vascular system, but may be applied to any anastomoses between tubular or hollow structures in the human or animal body. Any use of the term “vascular anastomosis” or “vascular anastomoses” is intended to refer to any anastomoses except when the context clearly limits the meaning to anastomoses between structures in the vascular system.

The present invention has been described with reference to certain preferred and alternative embodiments that are intended to be exemplary only and not limiting to the full scope of the present invention as set forth in the appended claims.

What is claimed is:

1. A device for creating an end-to-side anastomosis between a graft vessel and a recipient vessel, comprising:
an extravascular member having a first opening;
an intravascular member having a second opening with an edge;
means for attaching an end of the graft vessel to said edge;
a handle connected to said extravascular member and to said intravascular member.

2. The vascular clip of claim 1, wherein said handle further comprises a resilient element whereby said extravascular member and said intravascular member are biased together.

3. The vascular clip of claim 2, wherein said resilient element comprises a spring.

4. The vascular clip of claim 2, wherein said handle further comprises a pair of plates biased apart by said resilient element whereby a force applied between said plates moves said extravascular member and said intravascular member apart against the biasing action of said resilient element.

5. The vascular clip of claim 4, wherein each of said pair of plates comprises an indentation.

6. The vascular clip of claim 1, wherein at least one of said extravascular member and said intravascular member comprises a ring.

7. The vascular clip of claim 6, wherein said ring comprises an upwardly extending lip.

8. The vascular clip of claim 6, wherein said ring comprises an outwardly extending lip.

9. The vascular clip of claim 6, wherein said ring comprises a shape conformed to the shape of the recipient vessel.

10. The vascular clip of claim 1, wherein at least one of said extravascular member and said intravascular member comprises a hemi-cylinder.

11. The vascular clip of claim 1, wherein said extravascular member comprises a hemi-cylinder and said intravascular member comprises an expandable cylinder.

12. The vascular clip of claim 1, wherein said intravascular member comprises a scroll-like body having an inner longitudinal edge disposed in an interior of said scroll-like body and an outer longitudinal edge disposed to the exterior of said scroll-like body; and wherein at least one wire is attached to said inner longitudinal edge and through a wall of said scroll-like body to said extravascular member and wherein at least one wire is attached to said outer longitudinal outer edge and around said scroll-like body to said extravascular member.

13. The vascular clip of claim 1, further comprising a graft vessel and wherein said means for attaching an end of the graft vessel to said edge comprises suturing said end to said intravascular member.

14. The vascular clip of claim 13, wherein said graft vessel is an autologous graft.

15. The vascular clip of claim 13, wherein said graft vessel is a heterologous graft.

16. The vascular clip of claim 13, wherein said graft vessel comprises a synthetic material.

17. A device for creating an end-to-side anastomosis between a graft vessel and a recipient vessel, comprising:
an intravascular member having an opening with an edge;
means for attaching an end of the graft vessel to said edge; and
means for affixing said intravascular member within the recipient vessel.

18. The device of claim 17, wherein said intravascular member comprises an expandable cylinder.

19. The device of claim 18, where said expandable cylinder comprises a solid surface.

20. The device of claim 18, where said expandable cylinder comprises a mesh surface.

21. The device of claim 17, wherein said intravascular member comprises a scroll-like body having an inner longitudinal edge disposed in an interior of said scroll-like body and an outer longitudinal edge disposed to the exterior of said scroll-like body, further comprising means for compressing said intravascular member.

22. The device of claim 18, wherein said means for affixing said intravascular member within the recipient vessel comprises an inflatable balloon catheter disposed within said expandable cylinder.

23. The device of claim 18, further comprising means for inserting said intravascular member through a side of the recipient vessel.

24. The device of claim 23, wherein said means for inserting said intravascular member through a side of the recipient vessel comprises a boat-shaped delivery needle comprising a posterior portion for receiving said expandable cylinder, an anterior portion with a boat-shaped cross-section with sharpened upper edges for cutting an arteriotomy in the recipient vessel and means for discharging said expandable cylinder from said posterior portion and over said anterior portion through said arteriotomy.

25. The device of claim 23, wherein said means for inserting said intravascular member through a side of the recipient vessel comprises a boat-shaped delivery needle having sharpened upper edges for cutting an arteriotomy in the recipient vessel and an open interior for receiving said expandable cylinder.

26. A device for creating an end-to-side anastomosis between a graft vessel and a recipient vessel, comprising:
a graft vessel having a first end and a second end and a branch disposed between said first end and said second end,
a first intravascular member comprising a first expandable cylinder having a first opening with a first edge attached to said first end and a second intravascular member comprising a second expandable cylinder having a second opening with a second edge attached to said second end;
a first inflatable balloon catheter having a first tube extending through said graft vessel and exiting though said branch, said first balloon catheter disposed within said first expandable cylinder; and
a second inflatable balloon catheter having a second tube extending through said graft vessel and exiting though said branch, said second balloon catheter disposed within said second expandable cylinder.

27. An arteriotomy needle for making an arteriotomy in a recipient vessel comprising a boat-shaped body having sharpened upper edges for cutting an arteriotomy in a recipient vessel
